(19) United States
(12) Patent Application Publication
(10) Pub. No.: US 2020/0268432 A1
(43) Pub. Date: Aug. 27, 2020

(54) MEDICAL DEVICE AND MEDICAL DEVICE SYSTEM

(71) Applicant: OLYMPUS WINTER & IBE GMBH, Hamburg (DE)

(72) Inventors: Stefan DIETRICH, Berlin (DE); Daniel RAMIN, Nuthetal (DE)

(73) Assignee: OLYMPUS WINTER & IBE GMBH, Hamburg (DE)

(21) Appl. No.: 16/783,489

(22) Filed: Feb. 6, 2020

(30) Foreign Application Priority Data
Feb. 25, 2019 (DE) 102019104707.2

(51) Int. Cl. A61B 18/12

(52) U.S. Cl. CPC A61B 18/1206; A61B 2017/00199

(57) ABSTRACT

A medical device includes a medical function unit, an electronic control unit, and a voltage supply for the medical function unit and/or the electronic control unit, wherein the voltage supply is configured to be connected to a wired external supply voltage network, and wherein the electronic control unit includes a data interface in order to exchange data with an external device. The medical device wherein the data interface is configured to transmit and/or to receive data via at least one conductor of the supply voltage network. Furthermore, a medical device system is included.

### CROSS REFERENCE TO RELATED APPLICATION

This application claims the benefit of Germany Patent Application No. 102019104707.2, filed Feb. 25, 2019, which is hereby incorporated by reference herein in its entirety.

### BACKGROUND

The present invention relates to a medical device, comprising a medical function unit, an electronic control unit, and a voltage supply for the medical function unit and/or the electronic control unit, wherein the voltage supply is configured to be connected to a wired external supply voltage network, and wherein the electronic control unit comprises a data interface in order to exchange data with an external device.

Furthermore, the present invention relates to a medical device system comprising such a medical device.

Generic medical devices are indispensable in modern medicine. In addition to a function unit which provides the actual medical functionality, they have an electronic control unit which assumes a variety of functions. In addition to controlling the actual medical function unit, the electronic control assumes, inter alia, the communication to the user via a user interface, and possibly the communication with other medical devices and with patient data management systems.

In order to maintain the functional readiness of such medical devices, the electronic control unit must be maintained regularly. For this reason, the electronic control unit is connected to an external device, for example, a maintenance device, via a data interface, in order to exchange data with said device. The data may be operating or diagnostic data of the medical device which may be evaluated for detecting or analyzing malfunctions by the maintenance device. Furthermore, the data may be modified configuration data via which the functionality of the medical device can be adjusted. Likewise, the data may be new or modified operating software which is to be executed by the electronic control unit. The data interface may, for example, be configured as a USB or Ethernet interface.

For various reasons, an open data interface is not unproblematic. On the one hand, such a data interface constitutes a safety risk, since theoretically, unauthorized access to the electronic control unit of the medical device is made possible over it. Such access may comprise reading out confidential patient data or configuration data. Likewise, via unauthorized access, the configuration or the operating software of the electronic control unit may be manipulated, possibly resulting in a failure or a malfunction.

An additional problem is that an open interface is difficult to clean and thus constitutes a contamination risk.

The described problems were heretofore handled, inter alia, in that the data interface of a medical device was provided in the interior of a closed housing of the medical device. In order to access the interface, the medical device preferably had to be removed from an operating room in order to open the housing and connect the maintenance device. While the described problems have been largely eliminated by means of the described design of a medical device, the handling of a corresponding medical device during the maintenance is difficult. Simultaneously, the operation of a medical device with an opened housing constitutes an accident hazard for the operator, since live parts, including parts carrying a high voltage, may lie open. In addition, returning a medical device to service after temporarily opening the housing requires a comprehensive functional test.

### SUMMARY

The object of the present invention is therefore to provide a medical device and a medical device system which are improved with respect to the described problems.

This object is achieved according to one aspect of the present invention by a medical device, comprising a medical function unit, an electronic control unit, and a voltage supply for the medical function unit and/or the electronic control unit, wherein the voltage supply is configured to be connected to a wired external supply voltage network, and wherein the electronic control unit comprises a data interface in order to exchange data with an external device which is refined in that the data interface is configured to transmit and/or receive data via at least one conductor of the supply voltage network.

By means of the corresponding configuration of the data interface, in addition to a connection device for connecting to the supply voltage network, which must be present anyway on the medical device, no other connection devices are required on the medical device, whereby both the data security and the cleanability of the medical device are improved. This statement relates exclusively to data interfaces. The medical device may have other interfaces to which applicators or other instruments may be connected.

In a possible implementation of a medical device according to the present invention, the data interface may comprise a modulation and/or demodulation device, by means of which a data signal can be modulated onto a supply signal of the supply voltage network, and/or a data signal modulated onto the supply signal of the supply voltage network can be demodulated from said supply signal. In this way, the data signal may be transmitted independently of a network frequency possibly predominating in the supply voltage network.

The modulation type and the modulation frequency may be chosen according to the desired bandwidth and range of the data transmission. In this case, a higher data rate may be transmitted at a high modulation frequency, wherein a higher attenuation of the signal in the conductors of the supply voltage network which are not designed for high frequency takes place simultaneously, so that only small distances can be bridged. On the other hand, if greater distances are to be bridged, a lower carrier frequency can be chosen, wherein only a lower data rate is available.

In an advantageous refinement of a medical device according to the present invention, the modulation and/or demodulation device comprise at least one frequency filter. The frequency filter can separate the network frequency, which, for example, may be at approximately 50 Hz, from the modulation frequency, which, for example, may be at several 100 kHz.

In one embodiment of the present invention, the data interface of a medical device may comprise a memory device in order to temporarily store data received or to be transmitted via the data interface. In comparison to dedicated data networks, in the case of transmission of data via a supply voltage network, significantly more interference signals must be expected due to additional connected loads.

In order to ensure a smooth exchange of the data with the electronic control unit, here, data to be transmitted by the medical device are initially transmitted into the memory device in a data transfer which is fast and not very susceptible to interference, and then output by the data interface according to available bandwidth via the supply voltage network. Reciprocally, data to be received by the medical device is first stored in the memory device. As soon as the data to be received are completely stored in the memory device, said data is completely transmitted to the electronic control unit.

In order to reduce the risk of data errors due to interference signals, it is recommended to use a suitable error-detecting or error-correcting data protocol for data transmission.

The data to be transmitted via the data interface comprise operating data and/or diagnostic data. The data to be received via the data interface comprise configuration data and/or operating software.

In the case of a medical device according to a particularly preferable embodiment of the present invention, the data interface may be configured to encrypt data before the transmission and/or to decrypt data after the reception. Encrypting the data is expedient, since the data transmission through the supply voltage network can be easily spied on, for example, via a power outlet in publicly accessible areas of a hospital in which the medical device is used. In particular when transmitting confidential data, for example, personality-related patient data, encryption is thus desirable. For the encryption, in principle, any known symmetrical or asymmetrical encryption protocols may be used.

In a possible embodiment of the present invention, the data interface may be configured to transmit data in response to a request signal.

The medical function unit of a medical device according to the present invention may be configured for carrying out various medical functions. The medical device may be an irrigation pump, an insufflator, a video control unit, or an ultrasound generator. In a preferred embodiment of the present invention, the medical device may be an electrosurgical generator.

According to a further aspect of the present invention, the object is furthermore achieved via a medical device system, comprising a medical device according to the above embodiments, and a maintenance device which is configured to be connected to a wired supply voltage network, wherein the maintenance device comprises a data interface which is configured to exchange data with the medical device via at least one conductor of the supply voltage network.

According to an additional aspect of the present invention, the object is furthermore achieved via a medical device system, comprising: a first medical device and at least a second medical device, wherein the first medical device and the at least one second medical device are respectively designed and configured according to the present invention to exchange data with each other.

With respect to the effects and advantages which are hereby achievable, reference is made to what has already been stated above.

Within the scope of the present invention, the term “medical function unit” is to be interpreted in such a way that it relates to a logical and/or physical unit which provides the main function of the medical device. This main function does not necessarily have to be a direct medical function; for example, a video monitor to be considered to be a medical device may comprise the medical function unit assemblies for receiving, decoding, and depicting a video signal, even if these assemblies are likewise present in non-medical video monitors.

The medical function unit and the electronic control unit do not necessarily have to be separate components of a medical device according to the present invention, but may also partially or completely comprise identical components.

### BRIEF DESCRIPTION OF DRAWINGS

FIG. 1 depicts an example of a medical device system connected to a supply voltage network;

FIG. 2 depicts an example of two medical devices.

### DETAILED DESCRIPTION

The present invention will be described in greater detail below based on several figures, wherein the figures and exemplary embodiments are to contribute solely to a better understanding of the present invention, without limiting it.

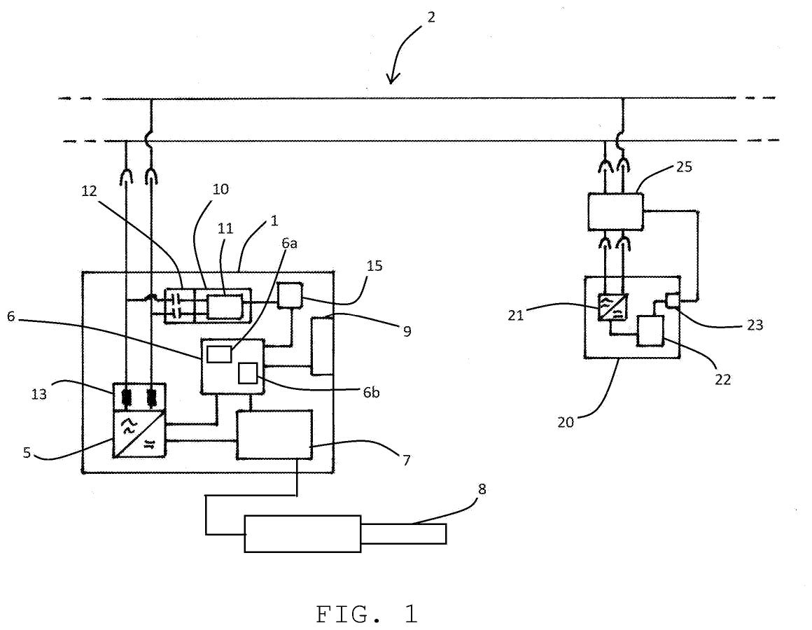

FIG. 1 depicts a medical device system comprising a medical device 1 which is connected to a supply voltage network 2. The supply voltage network is depicted as a two-wire network (phase+neutral conductor), but may also be designed having several wires (3 phases+neutral conductor), or five wires (3 phases+neutral conductor+protective conductor).

The medical device 1 furthermore has a voltage supply 5 in the form of a power supply, an electronic control unit 6, and a medical function unit 7. In the depicted example, an instrument 8 is connected to the medical function unit 7.

The electronic control unit 6 controls the function of the medical function unit 7, and simultaneously provides a user interface 9 via which a user can use the medical device 1. The user interface 9 may include a touchscreen, and/or may have separate operating elements such as rotating actuators or switches. The user interface 9 may also comprise external operating elements, for example, foot switches (not depicted).

Depending on the type of the medical device 1, the medical function unit 7 provides one or multiple medical functionalities. The medical device 1 may, for example, be or comprise an electrosurgical generator, an insufflator, an endoscope control device, an ultrasound generator, or an irrigation pump.

The control unit 6 comprises a microprocessor 6a and a memory unit 6b, in which program commands for the microprocessor 6a and configuration data of the medical device 1 are stored. In addition, during the operation of the medical device 1, the controller 6 ascertains operating and diagnostic data which are likewise stored in the memory unit 6b. In order to maintain the operational readiness of the medical device 1, the control unit 6 must be maintained; in this case, operating and/or diagnostic data are read out, and possibly updated program commands and/or configuration data are stored in the memory unit 6b.

In order to enable the data communication required within the scope of the maintenance, the medical device 1 has a data interface 10. The data interface 10 is configured to transmit and/or to receive data via at least one conductor of the voltage supply network 2. For this purpose, the data interface has a modulation and/or demodulation device (modem) 11 which modulates a data signal to the a high-frequency carrier frequency for transmitting data, and feeds the resulting signal into the conductors of the supply voltage network 2. For receiving data, the modem 11 taps the carrier frequency having the data signal from the conductors of the supply voltage network 2, and subsequently separates the data signal from the carrier frequency.

Frequency filters 12, 13 are provided in order to separate the carrier frequency and a base frequency of the supply voltage network 2. The filter 12 is a component part of the modem 11, while the filter 13 is a protective filter of the voltage supply 5.

For the data transmission via the supply voltage network 2 described here, two conductors of the supply voltage network 2 are used. However, data transmission methods are also conceivable which use only one or more than two conductors. Likewise, the number of conductors of the supply voltage network 2 used for the data transmission may vary from the total number of conductors of the supply voltage network 2.

The data interface 10 furthermore comprises a memory device 15 for temporarily storing received data and/or data to be transmitted.

The data interface 10 may be provided in the medical device 1 as a separate assembly. Alternatively, the data interface may be arranged completely or partially together with other components on a printed circuit board, for example, together with the control unit 6. In principle, it is also possible to arrange the data interface 10 outside a housing of the medical device 1.

The medical device system furthermore comprises a maintenance device 20 which is likewise connected to the supply voltage network 2. The maintenance device 20 comprises a power supply 21 and a maintenance unit 22 which, for example, is designed as a PC. The maintenance device has a standardized data interface 23 which may be a USB or Ethernet interface. In order to exchange data with the medical device 1 via the supply voltage network 2, the data interface 23 of the maintenance device 20 is connected via an adapter 25 to the supply voltage network 2, said adapter being configured similarly to the data interface 10.

When transmitting data via the conductors of the supply voltage network 2, interference signals may occur which, for example, result from switching actions or interference of other loads connected to the supply voltage network. In the case of electrosurgical instruments, these interference signals may considerably impair data transmission. It is therefore advantageous to select an error-tolerant transmission protocol when transmitting the data.

Due to the possible interference with the data transmission, the transmission speed may be low. In order not to block the control unit 6 too long, the data interface 10 is equipped with a separate memory device 15. Data which are received or which are to be transmitted are stored temporarily in the memory device 15. If data are to be transmitted by the control unit 6, these data are initially completely transmitted in a rapid internal transfer into the memory device 15. Thereafter, the data are transmitted to the maintenance device 20, depending on the available transmission rate. Likewise, received data are stored by the data interface 10 in the memory device 15, and are completely transmitted to the control unit 6 only after the completed transmission of all data.

In this way, the control unit 6 is preferably used to capacity as little as possible, so that it can continue to control the medical function unit 7. Nonetheless, the transmission of larger data quantities should preferably take place when the medical device 1 is not in operation.

Generally, the communication between the medical device 1 and the maintenance device 20 is controlled by the maintenance device 20. This means that the data interface 10 of the medical device 1 is inactive until the maintenance device 20 transmits an activation signal via the supply network 2. The activation signal may, for example, contain an identification number of the data interface 10, so that only the correct data interface is activated, if several medical devices are connected to the voltage supply network 2. The activation signal may furthermore contain a piece of information about whether the data interface 10 is to transmit or receive data, and which data are involved.

In particular updated operating software (updates, bug fixes, new features, etc.) or configuration data (parameters for new operating modes or instruments, etc.) are considered to be data to be received. In particular operating data (operating hour counters, procedure logs, etc.) or diagnostic data (error reports, internal measurement data, etc.) are considered to be data to be transmitted by the data interface 10. Since these data are in part confidential, an encrypted data transmission protocol is normally used, wherein symmetrical and asymmetrical encryptions are equally possible.

The use of the voltage supply network 2 as a medium for data transmission makes the medical device system particularly flexible. Thus, the medical device 1 and the maintenance device 20 can be arranged together in a separate low-interference system, for example, in a fabrication or maintenance center of the manufacturer of the devices. In such a low-interference system having short transmission paths, it is possible to achieve high data rates in order, for example, to carry out an initial installation having extensive operating software and/or configuration data, or in order to read out operating data of commissioning tests.

However, in the field, the medical device 1 is often integrated into an operation installation. Here, the maintenance device may then be connected to a freely accessible outlet of the supply voltage network 2, which may be either in the same room or in another room. Unlike in the previously described use case, more interference would have to be taken into account here, since other medical devices are possibly in operation in adjacent operating rooms which are connected to the same voltage supply network. Since, however, the data quantities to be transmitted in the field are generally lower than the data quantities at the initial installation, a sufficient transmission speed may also be achieved in this environment.

For the technical implementation of the present invention, use may be made of technologies known in principle from the field of carrier frequency systems. A detailed description is therefore omitted here for reasons of clarity.

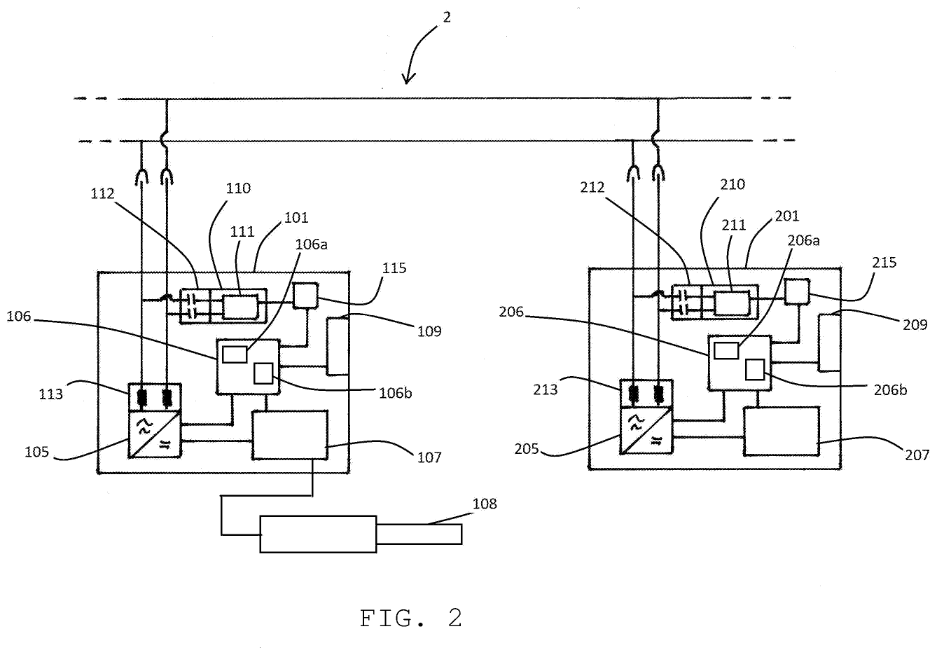

FIG. 2 depicts an additional medical device system which comprises two medical devices 101, 201. The basic structure of the medical devices 101, 201 corresponds to that of the medical device 1 from FIG. 1. Corresponding elements are therefore are provided with a reference character increased by 100 or 200 and are not described again.

The medical devices 101, 201 are configured to exchange data via the supply voltage network 2. The data exchange functions in principle exactly like the data exchange between the medical device 1 and the diagnostic device 20 in FIG. 1.

The medical device 101 may in turn be an electrosurgical generator; the medical device 201 is preferably provided for another medical functionality. For example, the medical device 201 may be an auxiliary device which carries out a function supporting the electrosurgical generator 101, for example, flue gas extraction or a coolant pump. The data transmission between the medical devices 101, 201 can then serve to synchronize the function of the auxiliary device 201 with the function of the electrosurgical generator 101, i.e., for example, activating flue gas extraction or coolant supply when the electrosurgical generator 101 delivers energy via the instrument 108.

The medical device 201 may, for example, also be a video monitor on which the image of a room camera, a boom arm camera, or an endoscopic camera is depicted during a procedure. The medical device 101 can then transmit operating data such as the operating mode, activation status, or installed parameters via the supply voltage network 2 to the video monitor 201, which can then superimpose the received operating data as an overlay into the video image.

The medical device 201 may also be a medical device controller which provides a central operating platform in order to control a plurality of other medical devices. In this case, operating data may be transmitted from the medical device 101 to the medical device controller via the supply voltage network 2; control commands may be transmitted in parallel from the medical device controller to the medical device 101.

In the medical device system depicted in FIG. 2, the particular feature occurs that data transmission via the supply voltage network 2 is also desirable when the medical devices 101, 201 are activated. In particular in the case of medical devices such as electrosurgical generators, it is to be expected that considerable interference signals occur, which are coupled via the voltage supply 105 into the supply voltage network 2. On the one hand, these may be high-frequency interference voltages from the high-frequency output signal of the electrosurgical generator; on the other hand, distortions of the voltage profile of the supply voltage network 2 may take place due to a strongly fluctuating power current consumption of the voltage supply 105, for example, in the case of pulsed operating modes of the electrosurgical generator.

Various methods are available in order to enable stable data transmission, despite the described interference signals. An interfering effect of high-frequency interference signals may thus be prevented in that a carrier frequency selected for the data transmission is clearly outside, preferably below, a base frequency of the interference signals. Thus, for example, in the case of interference signals in the frequency range of several hundred kHz, a transmission frequency in the range of several 10 kHz, for example, 30 kHz, may be used. Such a transmission frequency is sufficient to transmit only operating and/or status data between the medical devices 101, 201. Furthermore, in particular in the case of pulsed operating modes of an electrosurgical generator, the data transmission may be limited to the pulse rest intervals.

Phase distortion in the supply voltage network 2 may be compensated for in that the data transmission is limited to phase segments of a network base frequency in the supply voltage network 2 which are not highly distorted. Likewise, an influence of the phase profile of the supply voltage network 2 may be reduced by means of suitable measures for power factor correction, for example, by means of the use of power factor correction filters.

1. A medical device, comprising:
a medical function unit,
an electronic control unit, and
a voltage supply for the medical function unit and/or the electronic control unit,
wherein the voltage supply is configured to be connected to a wired external supply voltage network, and
wherein the electronic control unit comprises a data interface in order to exchange data with an external device,
wherein the data interface is configured to transmit and/or receive data via at least one conductor of the supply voltage network.

2. The medical device as claimed in claim 1, wherein the data interface comprises a modulation and/or demodulation device, by means of which a data signal can be modulated onto a supply signal of the supply voltage network, and/or a data signal modulated onto the supply signal of the supply voltage network can be demodulated from said supply signal.

3. The medical device as claimed in claim 2, wherein the modulation and/or demodulation device comprises at least one frequency filter.

4. The medical device as claimed in claim 1, wherein the data interface comprises a memory unit in order to temporarily store data which are received or which are to be transmitted via the data interface.

5. The medical device as claimed in claim 1, wherein the data to be transmitted via the data interface comprise operating data and/or diagnostic data.

6. The medical device as claimed in claim 1, wherein the data to be received via the data interface comprise configuration data and/or operating software.

7. The medical device as claimed in claim 1, wherein the data interface is configured to encrypt data before the transmission and/or to decrypt data after the reception.

8. The medical device as claimed in claim 1, wherein the data interface is configured to transmit data in response to a request signal.

9. The medical device as claimed in claim 1, wherein the medical device is an electrosurgical generator.

10. A medical device system, comprising:
a medical device as claimed in claim 1, and
a maintenance device which is configured to be connected to a wired supply voltage network,
wherein the maintenance device comprises a data interface which is configured to exchange data with the medical device via at least one conductor of the supply voltage network.

11. A medical device system, comprising:
a first medical device, and
at least a second medical device,
wherein the first medical device and the at least one second medical device are respectively designed and configured according to claim 1, to exchange data with one another.
